(19) United States
(12) Patent Application Publication
(10) Pub. No.: US 2019/0254787 A1
(43) Pub. Date: Aug. 22, 2019

(54) METHOD FOR MANUFACTURING CUSTOMIZED ARTIFICIAL TOOTH AND CUSTOMIZED ARTIFICIAL TOOTH

(71) Applicant: THE CATHOLIC UNIVERSITY OF KOREA INDUSTRY-ACADEMIC COOPERATION FOUNDATION, Seoul (KR)

(72) Inventor: Sang Hwa LEE, Seoul (KR)

(21) Appl. No.: 16/314,563

(22) PCT Filed: Dec. 8, 2016
(86) PCT No.: PCT/KR2016/014354

§ 371 Date: Dec. 31, 2018

(30) Foreign Application Priority Data
Jul. 29, 2016 (KR) 10-2016-0097445
Dec. 8, 2016 (KR) 10-2016-0166307

(51) Int. Cl. A61C 13/08; A61C 13/00; A61C 8/00

(52) U.S. Cl. CPC A61C 13/08; A61B 2034/105; A61C 8/0018; A61C 13/0004

(57) ABSTRACT

Provided is a method of manufacturing a customized artificial tooth and a customized artificial tooth. A method of manufacturing a customized artificial tooth includes acquiring image information on an image of an alveolar bone and a tooth using computed tomography (CT) scanning, wherein slope information generated according to inclination degrees of a crown part and a root part of a tooth in an XYZ three-dimensional (3D) space is acquired together with image information such as root shape information of a tooth in which an implant is to be implanted; generating output modeling data in which a screw groove is formed, such that the screw groove in which the slope information is reflected is formed in the implant to be implanted in a tooth-extracted part of the tooth; and manufacturing the implant in which the screw groove is formed, using a 3D printer based on the output modeling data.

### TECHNICAL FIELD

The present disclosure relates to a method of manufacturing a customized artificial tooth and a customized artificial tooth, and more particularly, to a method of manufacturing a customized artificial tooth and a customized artificial tooth, which may enable manufacture of a customized implant in which an arrangement state or formation angle of a tooth and a shape of a natural tooth are reflected and forming of a screw groove that allows use of commercially available abutments (ready-made products) and screws in the implant, and may enable manufacture of an artificial tooth that allows the reproduction of a natural tooth and firm coupling in an oral cavity when a tooth has a plurality of roots.

### BACKGROUND ART

In general, an artificial tooth used to restore a damaged tooth includes an implant, an abutment, and a prosthesis.

Here, an implant refers to a fixed body that is implanted into an alveolar bone such that a prosthesis configured to restore a damaged tooth is supported by the alveolar bone. The abutment refers to a pole that is combined with the implant for connection between the implant and the prosthesis. The prosthesis is manufactured in a similar form to a patient's tooth.

It has been commonly known that a technique of manufacturing an artificial tooth having the above-described structure using a three-dimensional (3D) printer is lately widely used to thereby make it easy to manufacture a patient-customized artificial tooth.

However, according to a method of manufacturing an artificial tooth of the related art, since research and development are concentrated only on the design of a patient-customized structure, an implant, an abutment, and a prosthesis are easily manufactured individually. However, there has been no review of compatibility between components, such as a review of whether it is possible to design a structure capable of using an existing standardized abutment during the manufacture of an implant.

That is, according to a method of the related art in which an implant is molded using a 3D printer, detailed data on locations of a root part and a crown part of a tooth in an XYZ 3D space is not reflected. Simply, a screw groove to be combined with an abutment is uniformly formed at an implant, and occlusion and angles between components are implemented by adjusting an implanted position of the implant and shapes of the abutment and a prosthesis. Thus, there has been a problem in that commercially available ready-made abutments and screws cannot be used, and newly standardized abutments and screws should be separately manufactured.

In addition, according to the manufacturing method of the related art, when a plurality of tooth roots are provided, only one standard implant is implanted into a tooth-extracted part, and an artificial tooth is formed by connecting a prosthesis to the implanted implant. Accordingly, a hollow space is relatively largely formed in the tooth-extracted part, and the prosthesis is manufactured in a larger form than the implant. As a result, a natural tooth cannot be reproduced, and coupling force of the manufactured artificial tooth in the oral cavity is weakened.

### DESCRIPTION OF EMBODIMENTS

### Technical Problem

Accordingly, the present disclosure has been devised to solve the above-described problems. Provided is a method of manufacturing a customized artificial tooth, which may obtain relative slope information between a crown part and a root part of a tooth along with shape information of the tooth, and customize a screw groove of an implant based on the slope information, so that the implant may be manufactured by reproducing a root part of a natural tooth, and commercially available ready-made abutments and screws may be used.

Provided is a method of manufacturing a customized artificial tooth, which may enable manufacture of an implant having a shape closet to a shape of a tooth-extracted part (or a hollow space formed after a tooth is extracted) when a tooth has a plurality of roots, so that a natural tooth may be reproduced and an artificial tooth, which may firmly remain in an oral cavity, may be customized using a 3D printer.

Provided is a customized artificial tooth, which may reproduce a root part of a natural tooth as it is and enable the use of commercially available ready-made abutments and screws.

### Solution to Problem

According to an aspect of the present disclosure, a method of manufacturing a customized artificial tooth includes acquiring image information on an image of an alveolar bone and a tooth using computed tomography (CT) scanning, wherein slope information generated according to inclination degrees of a crown part and a root part of a tooth in an XYZ three-dimensional (3D) space is acquired together with image information including root shape information of a tooth in which an implant is to be implanted, generating output modeling data in which a screw groove is formed, such that the screw groove in which the slope information is reflected is formed in the implant to be implanted in a tooth-extracted part of the tooth, and manufacturing the implant in which the screw groove is formed, using a 3D printer based on the output modeling data.

According to another aspect of the present disclosure, a customized artificial tooth includes an implant configured to be implanted into a tooth-extracted part to reproduce a root part of a tooth and including a screw groove, wherein the screw groove is formed to have a slope according to an inclination degree between a crown part and the root part of the tooth in a three-dimensional (3D) space; a prosthesis configured to be connected to the implant to reproduce the crown part of the tooth; and an abutment located between the implant and the prosthesis, the abutment having one side combined with the screw groove and another side combined with the prosthesis to connect the implant with the prosthesis.

### Advantageous Effects of Disclosure

A method of manufacturing a customized artificial tooth having the above-described configuration according to the present disclosure can manufacture an implant and a screw groove configured to couple the implant with an abutment by using slope information between a crown part and a root part of a tooth along with shape information of the tooth. Thus, an artificial tooth, which enables the manufacture of a tooth according to a formation angle or an arrangement state and the use of commercially available ready-made abutments and screws, can be smoothly manufactured using a three-dimensional (3D) printer. As a result, a treatment time and treatment costs can be reduced, good-quality medical services can be provided, and a real natural tooth can be reproduced.

Furthermore, in an embodiment in which when a plurality of root parts of the tooth are formed and a plurality of tooth-extracted parts of the tooth are formed, image information including the plurality of tooth-extracted parts is acquired, a natural tooth identical to a real tooth can be reproduced, and components (e.g., an implant), which enable firm coupling in an oral cavity and a reduction in treatment time, can be manufactured rapidly and precisely.

In addition, a customized artificial tooth having the above-described configuration according to the present disclosure can enable the manufacture of a tooth according to a formation angle or an arrangement state and the use of ready-made abutments and screws by reflecting slope information between a crown part and a root part of a tooth in a screw groove of an implant. As a result, a treatment time and treatment costs can be reduced, good-quality medical services can be provided, and a real natural tooth can be reproduced.

### BRIEF DESCRIPTION OF DRAWINGS

FIG. 1 is a diagram showing an arrangement state of teeth, which is different according to person.

FIG. 2 is a diagram for explaining merits of an implant that is manufactured according to an embodiment of the present disclosure.

FIG. 3 is a diagram of a restoration structure for explaining a method of manufacturing a dental restoration according to another embodiment of the present disclosure.

FIG. 4 is a diagram of a restoration structure for explaining a method of manufacturing a dental restoration according to another embodiment of the present disclosure.

FIG. 5 is a diagram for explaining a process of modeling an abutment that is manufactured according to another embodiment of the present disclosure.

### BEST MODE

Hereinafter, a method of manufacturing a customized artificial tooth according to an embodiment of the present disclosure will be described in detail with reference to the accompanying drawings.

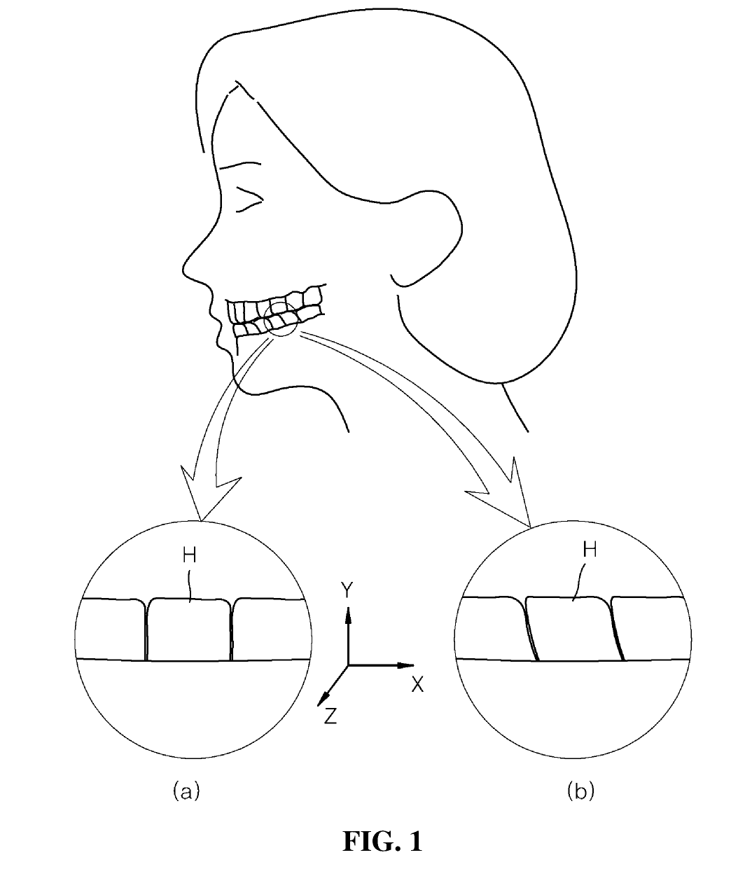

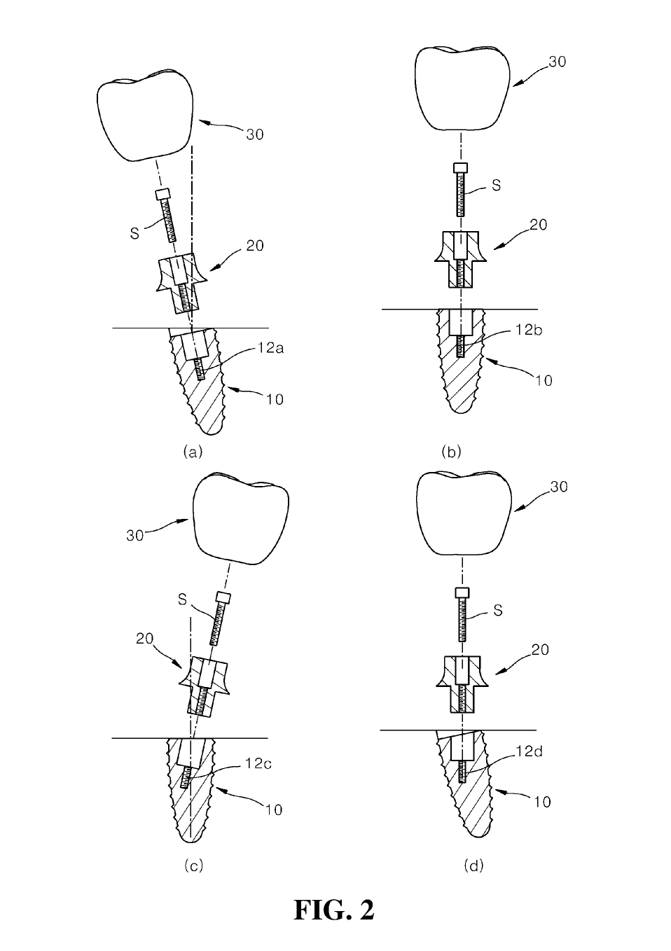

FIG. 1 is a diagram showing an arrangement state of teeth, which is different according to person, and FIG. 2 is a diagram for explaining merits of an implant that is manufactured according to an embodiment of the present disclosure.

As shown in FIGS. 1 and 2, a method of manufacturing a customized artificial tooth according to the present disclosure includes acquiring image information on an image of an alveolar bone and a tooth using a scanning unit, such as a computed tomography (CT), generating output modeling data (output by means of a three-dimensional (3D) printer) based on the image information, and manufacturing an implant shape using the 3D printer.

In the present disclosure, the operation of acquiring the image information includes acquiring image information on the image of the alveolar bone and the tooth. Slope information generated according to inclination degrees of a crown part and a root part of a tooth in an XYZ 3D space is acquired along with the image information.

That is, as shown in FIG. 1, since a shape of a jaw joint or an oral structure are different for each person, an arrangement state of teeth with respect to the gums is inevitably different. For example, as shown in portion (a) of FIG. 1, a person may have some teeth of which a crown part H is not inclined with respect to a Y-axis on an XY plane but arranged upright. As shown in portion (b) of FIG. 1, another person may have teeth of which a crown part H is inclined with respect to a Y-axis.

An arrangement state of teeth, which is different from person to person, will be described in further detail as follows.

That is, as shown in FIG. 2, arrangement states of teeth may be broadly classified into states shown in (a), (b), (c), and (d). (a) is an exploded view of an artificial tooth that is manufactured based on image data showing that a crown part and a root part of a tooth are inclined at the same angle about a Y-axis. (b) shows an artificial tooth that is manufactured based on image data showing that a crown part and a root part of a tooth are located on an axial line parallel to a Y-axis. (c) shows an artificial tooth that is manufactured based on image data showing that a root part of a tooth is located on an axial line parallel to a Y-axis and a crown part of the tooth is inclined about the Y-axis. (d) shows an artificial tooth that is manufactured based on image data showing that a root part of a tooth is inclined with respect to a Y-axis and a crown part of the tooth is located parallel to the Y-axis.

Meanwhile, an artificial tooth required for restoration of a damage tooth is typically divided into an implant 10, an abutment 20, and a prosthesis 30.

In the present disclosure, components of each artificial tooth are manufactured using a 3D printer. When the artificial tooth is manufactured, as shown in FIG. 2, it is important to manufacture the implant 10, the abutment 20, and the prosthesis 30 in various forms considering a different dental arrangement for each person. However, since one artificial tooth is formed by mutually combining the respective components, the availability of a commercially available ready-made product should be considered very important for patients or dentists.

As shown in FIG. 2, the operation of acquiring image information, which is adopted for the present disclosure, includes acquiring relative slope information on the crown part and the root part of the tooth along with image information on the tooth. Screw grooves 12a, 12b, 12c, and 12d, which are inclined at various angles with respect to the Y-axis, may be formed at the implant 10 based on the acquired information.

That is, the operation of generating the output modeling data, which is adopted for the present disclosure, includes forming the screw grooves 12a, 12b, 12c, and 12d in which the slope information is reflected, at an imaginary implant to be implanted into a tooth-extracted part of the tooth, based on the image information and the slope information of the tooth, and generating modeling data on the implant 10 at which the screw grooves 12a, 12b, 12c, and 12d are formed.

Here, in the present embodiment, the modeling data may be generated by reflecting preset standard information on the abutment 20, which is commercially available ready-made, and screw S along with the slope information.

Meanwhile, the screw grooves 12a, 12b, 12c, and 12d may be manufactured to use the commercially available ready-made abutment 20 and screw S as in the present embodiment or manufactured to use customized abutment and screw in some cases.

As described above, in the method of manufacturing the customized artificial tooth according to the present disclosure, the implant 10 and the screw grooves 12a, 12b, 12c, and 12d configured to couple the implant 10 with the abutment 20 may be manufactured using slope information between the crown part and the root part along with shape information of the tooth. Thus, the artificial tooth, which may be manufactured according to a formation angle or arrangement state of the tooth and use the commercially available ready-made abutment 20 and the screw S, may be smoothly manufactured using a 3D printer. As a result, a treatment time and treatment costs may be reduced, a good-quality medical service may be provided, and a real natural tooth may be reproduced.

In FIG. 2, an undescribed symbol S denotes a coupling screw, which passes through the abutment 20 and is fastened to the screw grooves 12a, 12b, 12c, and 12d.

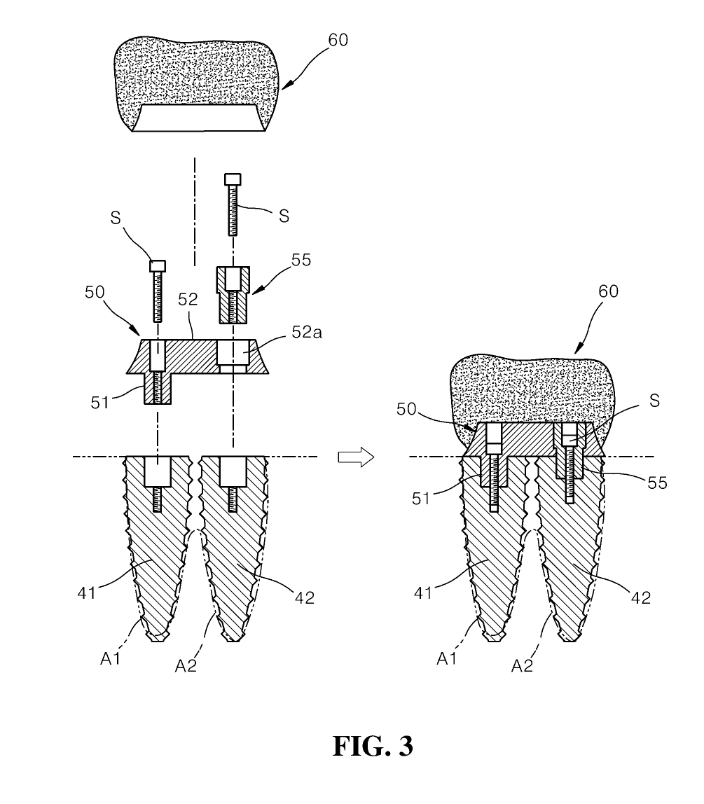

FIG. 3 is a diagram of an artificial tooth structure for explaining a method of manufacturing a customized artificial tooth according to another embodiment of the present disclosure.

The artificial tooth shown in FIG. 3 is used when a plurality of tooth-extracted parts of a tooth are formed due to the formation of a plurality of root parts of the tooth. The artificial tooth shown in FIG. 3 includes a pair of implants 41 and 42, which are individually implanted into tooth-extracted parts A1 and A2, respectively, one abutment 50, and one prosthesis 60.

The artificial tooth is manufactured using a method described below.

That is, the method of manufacturing the customized artificial tooth according to the present embodiment includes generating basic modeling data on shapes of implants 41 and 42 from image information on an alveolar bone and a tooth, generating output modeling data including image data on the abutment 50 along with the basic modeling data, and manufacturing the customized artificial tooth using a 3D printer based on the generated output modeling data.

In the present embodiment, when a tooth has a plurality of roots, basic modeling data capable of individually manufacturing a pair of implants 41 and 42 is generated based on image data on each of the tooth-extracted parts A1 and A2. Image data on the abutment 50 including a coupling portion 51 coupled to any one 41 of the implants 41 and 42 and an extension portion 52, which extends from the coupling portion 51 toward the another implant 42, is generated based on the basic modeling data.

The abutment 50 manufactured according to the present embodiment is not manufactured to have a structure corresponding one-to-one to the pair of implants 41 and 42, but is manufactured to include only one element that may be coupled to the pair of implants 41 and 42. However, to reinforce coupling force with the pair of implants 41 and 42, the extension portion 52 of the abutment 50 may be modeled to have a coupling groove 52a to which another abutment 55 is fastened.

As a result, in the present embodiment, basic modeling data on the implants 41 and 42 to be individually implanted into the respective tooth-extracted parts A1 and A2 is generated, and image data on the abutment 50 is generated based on the generated basic modeling data. Information on a size and position of the coupling groove 52a is acquired from the image data on the abutment 50, and image data on the extension portion 52 at which the coupling groove 52a is formed is generated based on the acquired information.

As described above, in the present embodiment, abutments 50 to be respectively fastened to the pair of implants 41 and 42 may not be individually manufactured. However, an abutment 50 coupled to only one 41 of the pair of implants 41 and 42 may be manufactured, and another abutment 55 fastened to another implant 42 through the abutment 50 may be then manufactured from image information on the abutment 50 and the implants 41 and 42. Thus, components of the artificial tooth, which may reinforce coupling force and shorten a treatment time, may be manufactured rapidly and precisely, and a natural tooth similar to a real tooth may be reproduced.

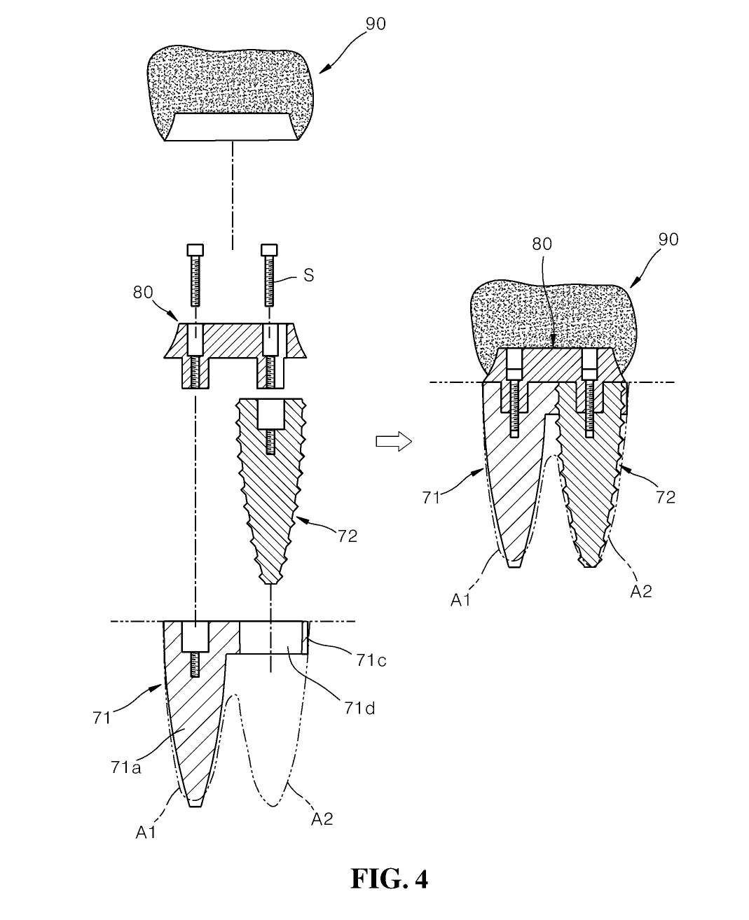

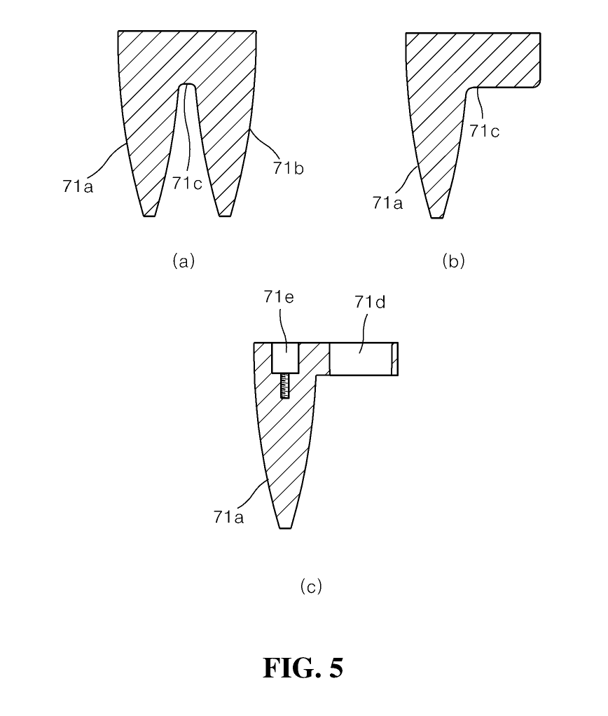

FIG. 4 is a diagram of an artificial tooth structure for explaining a method of manufacturing a customized artificial tooth according to another embodiment of the present disclosure. FIG. 5 is a diagram for explaining a process of modeling an abutment that is manufactured according to another embodiment of the present disclosure.

The artificial tooth shown in FIG. 4 is used when a pair of tooth-extracted parts A1 and A2 of a tooth are formed due to the formation of a plurality of root parts of the tooth. The artificial tooth shown in FIG. 4 includes a first implant 71, which is implanted into only one A1 of the tooth-extracted parts A1 and A2, a second implant 72, which passes through the first implant 71 and is implanted into another tooth-extracted part A2, one abutment 80, and one prosthesis 90.

The artificial tooth is manufactured using a method described below.

That is, as shown in FIG. 5, a method of manufacturing the customized artificial tooth according to the present embodiment includes generating basic modeling data including placement portions 71a and 71b having shapes corresponding to the pair of tooth-extracted parts A1 and A2 and a connecting portion 71c configured to connect the placement portions 71a and 71b, based on image information on an alveolar bone and a tooth (refer to (a) in FIG. 5), and removing any one 71b of the placement portions 71a and 71b from the basic modeling data and generating image data including only another placement portion 71a and the connecting portion 71c (refer to (b) in FIG. 5).

Furthermore, information on a size and a position of a fastening recess 71d capable of fastening the removed placement portion 71a to the connecting portion 71c and information on a screw groove 71e to be coupled to the abutment 80 are acquired from the image data and image information on the tooth. Image data on the fastening recess 71d and the screw groove 71e may be generated based on the acquired information (refer to (c) in FIG. 5).

As described above, in the present embodiment, when a tooth has a plurality of roots, the first implant 71 and the second implant 72 may be manufactured based on image data of the tooth-extracted parts A1 and A2. The first implant 71 includes the placement portion 71a, which is implanted into any one tooth-extracted part A1, and the connecting portion 71c, which extends toward another tooth-extracted part A2. The second implant 72 passes through the connecting portion 71c of the first implant 71 and is implanted into the another tooth-extracted part A2.

As described above, in the present embodiment, implants to be respectively fastened to the pair of tooth-extracted parts A1 and A2 are not individually manufactured, but the first implant 71, which is implanted into any one tooth-extracted part A1, and the second implant 72, which passes through the first implant 71 and is implanted into another tooth-extracted part A2, may be manufactured. Accordingly, an advantage of reproducing a crown part of the tooth and a crown connected to the crown part like a real natural tooth may be derived. Also, components (e.g., the implants 71 and 72) configured to enable firm coupling of the artificial tooth in the oral cavity and a reduction in treatment time may be manufactured rapidly and precisely.

Hereinafter, respective components of a customized artificial tooth according to an embodiment of the present disclosure will be described in detail with reference to FIG. 2.

As shown in FIG. 2, the customized artificial tooth according to the embodiment of the present disclosure includes an implant 10, an abutment 20, and a prosthesis 30.

The implant 10 is a portion to be implanted into a tooth-extracted part to reproduce a root part of a tooth. The implant 10 includes screw grooves 12a, 12b, 12c, and 12d, which are combined with the abutment 20.

The screw grooves 12a, 12b, 12c, and 12d may be formed to have slopes according to an inclination degree between a crown part and the root part of the tooth in a 3D space. Thus, as shown in (a), (b), (c), and (d) of FIG. 2, it is possible to manufacture the customized artificial tooth according to a formation angle or arrangement state, and it is possible to use the ready-made abutment 20 and the screw S.

The prosthesis 30 is a portion configured to reproduce a crown part of the tooth. The prosthesis 30 is connected to the implant 10 through the abutment 20.

The abutment 20 is configured to connect the implant 10 and the prosthesis 30. The abutment 20 is located between the implant 10 and the prosthesis 30, one side of the abutment 20 is combined with the screw grooves 12a, 12b, 12c, and 12d, and another side of the abutment 20 is combined with the prosthesis 30.

The abutment 20 is designed such that the screw grooves 12a, 12b, 12c, and 12d of the implant 10 are provided in view of a formation angle or an arrangement state of teeth, which is different according to each person. Thus, a commercially available model may be applied and in some cases, a customized model may be adopted instead of a ready-made model product.

The customized artificial tooth having the above-described configuration according to the present disclosure may be manufactured according to a formation angle or arrangement state of teeth so that slope information between the crown part and the root part may be reflected in the screw grooves 12a, 12b, 12c, and 12d of the implant 10. Also, the ready-made abutment 20 and the screw S may be used. As a result, a treatment time and treatment costs may be reduced, good-quality medical services may be provided, and a real natural tooth may be reproduced.

Meanwhile, slopes of the screw grooves 12a, 12b, 12c, and 12d may be acquired in various ways and reflected in the manufacture of the implant 10. However, image information on an image of an alveolar bone and a tooth is acquired using a scanning unit, such as a CT, and the slopes of the screw grooves 12a, 12b, 12c, and 12d are acquired based on the image information.

That is, formation angles (or slopes) of the screw grooves 12a, 12b, 12c, and 12d with respect to the implant 10 may be acquired based on slope information generated according to inclination degrees of the crown part and the root part of the tooth in an XYZ 3D space, along with the image information on the image of the alveolar bone and the tooth.

As shown in FIG. 3, the customized artificial tooth according to another embodiment of the present disclosure is used when a plurality of tooth-extracted parts A1 and A2 of a tooth are formed due to the formation of a plurality of root parts of the tooth. The customized artificial tooth shown in FIG. 3 includes a plurality of implants 41 and 42, an abutment (or main abutment) 50, an additional abutment (or sub-abutment) 55, and a fastening unit S.

The implants 41 and 42 are respectively implanted into the tooth-extracted parts A1 and A2 on a one-to-one basis.

The abutment 50 includes a coupling portion, which is coupled to any one 41 of the implants 41 and 42, and an extension portion 52, which extends from the coupling portion 51 toward another implant 42 and at which a coupling groove 52a is formed.

The another abutment 55 is inserted into a screw groove of the another implant 42 via the coupling groove 52a of the abutment 50 and is firmly coupled to the another implant 42 by a fastening unit such as a screw S.

The customized artificial tooth having the above-described configuration according to another embodiment of the present disclosure is configured such that any one implant 41 is coupled using the main abutment 50, and the main abutment 50 is coupled together with another implant 42 using the sub-abutment 55, instead of coupling a plurality of implants using one abutment. (That is, the customized artificial tooth is configured to flexibly cope with a formation angle of the tooth using the sub-abutment 55). Thus, it is possible to reinforce coupling force, shorten a treatment time, and reproduce a natural tooth similar to a real tooth.

As shown in FIG. 4, the customized artificial tooth according to yet another embodiment of the present disclosure is used when a pair of tooth-extracted parts A1 and A2 of a tooth are formed due to the formation of a plurality of root parts of the tooth. The customized artificial tooth shown in FIG. 4 includes a first implant 71 configured to be implanted only to any one A1 of the tooth-extracted parts, a second implant 72 configured to pass through the first implant 71 and be implanted into another tooth-extracted part A2, one abutment 80, and one prosthesis 90.

The first implant 71 includes a placement portion 71a, which is implanted in any one A1 of the tooth-extracted parts, and a connecting portion 71c, which extends from the placement portion 71a toward another tooth-extracted part A2 and at which a fastening recess 71d is formed.

The second implant 72 is implanted in the another tooth-extracted part A2 through the fastening recess 71d of the connecting portion 71c, and the abutment 80 is combined with the first implant 71 and the second implant 72.

In the present embodiment having the above-described configuration, implants to be respectively fastened to the pair of tooth-extracted parts A1 and A2 are not individually provided, but the customized artificial tooth includes the first implant 71, which is implanted into any one tooth-extracted part A1, and the second implant 72, which passes through the first implant 71 and is implanted into another tooth-extracted part A2. Thus, an advantage of reproducing a crown part of the tooth and a crown connected to the crown part like a real natural tooth and an advantage of enabling firm coupling of an artificial tooth in the oral cavity and a reduction in treatment time may be expected.

While various embodiments of the present disclosure have been described, the present embodiments and the appended drawings are intended to clearly describe and illustrate some of the technical ideas included in the present disclosure. It is obvious that modifications and specific embodiments, which may be easily understood by one of skill in the art within the scope of the technical ideas included in the specification and drawings of the present disclosure, fall within the scope and spirit of the present disclosure.

1. A method of manufacturing a customized artificial tooth, the method comprising:
acquiring image information on an image of an alveolar bone and a tooth using computed tomography (CT) scanning, wherein slope information generated according to inclination degrees of a crown part and a root part of a tooth in an XYZ three-dimensional (3D) space is acquired together with image information including root shape information of a tooth in which an implant is to be implanted;
generating output modeling data in which a screw groove is formed, such that the screw groove in which the slope information is reflected is formed in the implant to be implanted in a tooth-extracted part of the tooth; and
manufacturing the implant in which the screw groove is formed, using a 3D printer based on the output modeling data.

2. The method of claim 1, wherein when the tooth has a plurality of root parts and a plurality of tooth-extracted parts of the tooth are formed, basic modeling data on an implant to be individually implanted into each tooth-extracted part is generated from the image information,
wherein the generating of the output modeling data comprises generating image data on an abutment comprising a coupling portion coupled to any one of implants and an extension portion configured to extend from the coupling portion toward another implant, along with the basic modeling data.

3. The method of claim 2, wherein the generating of the output modeling data comprises acquiring information on a size and a position of a coupling groove configured to couple the extension portion of the abutment with the another implant from the image data on the abutment, and generating image data in which the coupling groove is formed in the extension portion, based on the acquired information.

4. The method of claim 1, wherein, when a plurality of root parts of the tooth are formed and a plurality of tooth-extracted parts of the tooth are formed, basic modeling data including placement portions having shapes corresponding to the plurality of tooth-extracted parts and a connecting portion configured to connect the placement portions is generated from the image information,
wherein the generating of the output modeling data comprises generating image data including only another one of the placement portions and the connecting portion and in which the screw groove is reflected, by removing any one of the placement portions from the basic modeling data.

5. The method of claim 4, wherein the generating of the output modeling data comprises acquiring information on a size and a position of a fastening recess configured to fasten the removed placement portion to the connecting portion, from the image data including only the another placement portion and the connecting portion and the image information on the tooth image, and generating image data in which the fastening recess is formed in the connecting portion, based on the acquired information.

6. A customized artificial tooth comprising:
an implant configured to be implanted into a tooth-extracted part to reproduce a root part of a tooth and comprising a screw groove, wherein the screw groove is formed to have a slope according to an inclination degree between a crown part and the root part of the tooth in a three-dimensional (3D) space;
a prosthesis configured to be connected to the implant to reproduce the crown part of the tooth; and
an abutment located between the implant and the prosthesis, the abutment having one side combined with the screw groove and another side combined with the prosthesis to connect the implant with the prosthesis.

7. The customized artificial tooth of claim 6, when a plurality of root parts of the tooth are formed and a plurality of tooth-extracted parts of the tooth are formed, further comprising:
a plurality of implants configured to be implanted into the tooth-extracted parts, respectively;
an abutment comprising a coupling portion coupled to any one of the implants and an extension portion extending from the coupling portion toward another implant and, wherein a coupling groove is formed in the extension portion;
another abutment configured to be inserted into a screw groove of the another implant through the coupling groove of the abutment; and
a fastening unit configured to pass through the another abutment and be fastened to the screw groove of the implant.

8. The customized artificial tooth of claim 6, when a plurality of root parts of the tooth are formed and a plurality of tooth-extracted parts of the tooth are formed, further comprising:
a first implant comprising a placement portion configured to be implanted into any one of the tooth-extracted parts and a connecting portion extending from the placement portion toward another tooth-extracted part, wherein a fastening recess is formed in the connecting portion;
a second implant configured to be implanted into the another tooth-extracted part through the fastening recess of the connecting portion; and
an abutment configured to be combined with the first implant and the second implant.
